(19) United States
(12) Patent Application Publication
(10) Pub. No.: US 2024/0299645 A1
(43) Pub. Date: Sep. 12, 2024

(54) ADHESIVE PAD WITH FEATURES TO AID APPLICATION AND HANDLING OF A MEDICAL DEVICE

(71) Applicant: INSULET CORPORATION, Acton, MA (US)

(72) Inventors: Ayden HENSON, Arlington, MA (US); Jeffrey BARNES, Medford, MA (US); Lucas MCGAHREN, Somerville, MA (US)

(21) Appl. No.: 18/600,948

(22) Filed: Mar. 11, 2024

Related U.S. Application Data
(60) Provisional application No. 63/489,249, filed on Mar. 9, 2023.

(51) Int. Cl. A61M 5/142; G16H 20/17

(52) U.S. Cl. CPC A61M 5/14248; G16H 20/17; A61M 2210/083; A61M 2210/086; A61M 2210/1021

(57) ABSTRACT

Described herein are adhesive drug delivery application devices and related systems, methods, and kits useful for applying a medical device (such as a drug delivery device or a transcutaneous or subcutaneous sensor) to an adhesion site on the skin of a user. A medical device application system may include a needle cap, an adhesive platform, and a temporary backing. The temporary backing may, for example, include a first wing and a second wing. The temporary backing may also include a tab. In other examples, the adhesive platform may include a first bridge and a second bridge, a temporary loop, and/or a first sticker and a second sticker.

### CROSS-REFERENCE TO RELATED APPLICATIONS

This application claims priority to and the benefit of U.S. Provisional Application No. 63/489,249, filed Mar. 9, 2023, the entirety of which is incorporated herein by reference.

### TECHNICAL FIELD

The present disclosure is related to devices, systems, methods, and kits for attachment devices of a wearable device enabling simpler attachment of the wearable device to a patient. More particularly, the disclosure describes examples of application devices for applying a drug delivery device to a patient.

### BACKGROUND

Many conventional drug delivery systems, such as handheld auto-injectors, are designed to rapidly deliver a drug to a patient. These conventional drug delivery systems are generally not suitable for delivering a drug to a user over relatively longer periods of time as may be required for many drugs.

As an alternative to conventional auto-injectors, there are drug delivery systems designed to be wearable (e.g., applied to the skin of a patient). To apply the drug delivery device to the skin, a patient grips the drug delivery device on either side of a small, often curved surface of the device which may be smaller than the average width of a patient's finger. The patient must remove a cover from the adhesive platform by pulling the cover away from the adhesive platform while tightly holding the device and applying the device to the skin.

Applying the drug delivery device requires dexterity and finger strength. Patients with limited finger strength and dexterity may experience challenges maintaining a grip on the drug delivery device potentially causing the patient to touch the adhesive surface or to drop the drug delivery device. Dropping the drug delivery device may render the device un-usable because it may impact mechanical features of the device. Touching the adhesive surface can contaminate it and adversely impact the ability of an adhesive surface to stick to the user. Further, drug delivery devices are becoming smaller, which makes handling the device more difficult and adhesive strength increasingly important to application of the device and operability.

A need therefore exists for a more convenient and user-friendly wearable drug delivery device that enables the drug delivery device to be more easily attached to the skin of a user.

### SUMMARY

In one aspect, embodiments described herein relate to a medical device application system for positioning a medical device on a user. In some embodiments, a medical device application system for positioning a medical device on a user is described that includes an adhesive platform device operable to be coupled to the medical device, the adhesive platform device having a first platform surface and a second platform surface, and a temporary backing detachably connected to the first platform surface of the adhesive platform, wherein the second platform surface of the adhesive platform is configured to interface with the wearable drug delivery device. In some embodiments, the temporary backing further comprises a first temporary backing wing extending from a first backing side of the temporary backing and a second temporary backing wing extending from a second side of the temporary backing, wherein the second backing side extends from the opposite side as the first backing side. In some embodiments, the medical device application system further comprises a temporary backing tab extending from the temporary backing. In some embodiments, the adhesive platform device further comprises, a first platform bridge extending from a first platform side of the temporary backing, and a second platform bridge extending from a second platform side of the temporary backing, wherein the second side is opposite the first side, and the adhesive platform bridges are paired with the temporary backing bridges. In some embodiments, the medical device is a drug delivery device. In some embodiments, the medical device is an analyte sensor, such as a continuous glucose monitor, or another type of analyte sensor. In some embodiments, the adhesive platform device is configured to adhere to skin of the user.

In an aspect of the disclosed subject matter, a medical device application system for positioning a medical device on skin of a user is also described that includes an adhesive platform device. The adhesive platform device may include a first platform surface and a second platform surface, a temporary backing detachably connected to the first platform surface. The second platform surface is coupled to the medical device, and a temporary loop detachably connected to an external facing surface of the medical device.

In another aspect of the disclosed subject matter, a medical device application system for positioning a medical device on a user is described that includes an adhesive platform device having a first platform surface and a second platform surface, a temporary backing detachably connected to the adhesive platform first surface, a first sticker having a first sticker adhesive side and a first sticker gripping side, and a second sticker having a second sticker adhesive side and a second sticker gripping side, wherein at least a first portion of the first sticker adhesive side and at least a first portion of the second sticker adhesive side are detachably connected to the medical device external facing surface, and a second portion of the first sticker adhesive side is detachably connected to a second portion of the second sticker adhesive side.

In yet another aspect of the disclosed subject matter, a medical device application system for positioning a medical device on a user is described that comprises an adhesive platform device comprising a first platform surface and a second platform surface, wherein the second platform surface is coupled to the medical device, a temporary backing detachably connected to the first platform surface, a temporary backing first wing having a first end detachably connected to the second platform surface, and a temporary backing second wing having a first end detachably connected to the second platform surface.

In a further aspect of the disclosed subject matter, a medical device application system for positioning a medical device on a user is described that include an adhesive platform device having a first platform surface and a second platform surface, wherein the second platform surface is coupled to the medical device, a temporary backing detachably connected to the first platform surface, and a moveable layer attached at least partially to the second platform surface.

### BRIEF DESCRIPTION OF THE DRAWINGS

FIGS. 1A-1D illustrate a first exemplary embodiment of a medical device application system in deconstructed and constructed views.

FIGS. 2A-2M illustrate various views of the first exemplary embodiment of the drug delivery device application for use with the wearable drug delivery device and an exemplary method of application.

FIG. 3 illustrates a second exemplary embodiment of the medical device application system.

FIG. 4 illustrates a third exemplary embodiment of a medical device application system including a temporary loop.

FIG. 5 illustrates a fourth exemplary embodiment of the wearable drug delivery device and an exemplary method of assembly of a drug delivery application device utilizing temporary attachments.

FIGS. 6A-6C illustrate various views of a fifth exemplary embodiment of the medical device application system including temporary backing wings.

FIG. 7 illustrates a patient facing isometric view of a sixth exemplary embodiment of the medical device application system including temporary wings.

FIGS. 8A-8C illustrate various views of a seventh exemplary embodiment of the medical device application system including temporary wings.

FIGS. 9A-9B illustrate various views of an eighth exemplary embodiment of the medical device application system including temporary wings.

FIG. 10A-10C illustrate various views of a first exemplary embodiment of the medical device application system including temporary handles.

FIG. 11 illustrates various views of a second exemplary embodiment of the medical device application system including temporary handles.

FIG. 12 illustrates a third exemplary embodiment of the medical device application system including temporary handles.

FIG. 13 illustrates a fourth exemplary embodiment of the medical device application system including temporary handles.

FIGS. 14A and 14B illustrate a further exemplary embodiment of a medical application system including an adhesive platform and a movable layer.

FIGS. 15A-15F illustrate various views of another exemplary embodiment of the medical device application system including temporary handles.

FIG. 16 illustrates various views of a yet another exemplary embodiment of the medical device application system including temporary handles.

FIG. 17 illustrates an additional exemplary embodiment of the medical device application system including temporary handles.

### DETAILED DESCRIPTION

This disclosure presents various systems, components, and methods related to an adhesive application device for a medical device. Each of the systems, components, and methods disclosed herein provides one or more advantages over traditional systems, components, and methods. Various embodiments of the adhesive application devices, medical device application systems, components, and methods are disclosed herein.

As used herein, the term “external facing surface” may refer to a surface of a medical device, such as a drug delivery device or another component having a surface facing away from the adhesion site or the region of patient's skin for which the drug delivery device is adhered. Embodiments described herein may use the terms “internal facing surface,” and “patient facing surface” interchangeably.

As used herein, the term “internal facing surface” may refer to the surface of a medical device or another component having a surface facing towards the adhesion site or the region of patient's skin for which the medical device, such as a drug delivery device, is adhered. Embodiments described herein may use the terms “external facing surface,” and “patient opposing surface” interchangeably.

Medical devices such as wearable drug delivery devices, analyte sensing devices (such as a continuous blood glucose monitor, ketone sensor, or the like), heart rate monitors, or the like that may be placed on the skin of a patient using an adhesive platform to attach the medical device to a site on the patient. A needle or sensor may be inserted from the device into the skin of the patient to enable the medical device to perform its intended function(s), such as delivering medication over time. The medical device remains adhered to the patient so, for example, medication can continue to be delivered to the patient or the sensor can perform its function(s). Gripping the medical device in a way that reduces the risk of dropping the medical device while preventing contamination of the adhesive platform is important to the long-term operation of the medical device and adherence of the medical device to the skin of the patient.

Various embodiments disclosed herein are directed towards medical device application devices that are configured to be held by a user of the medical device or a caregiver to hold the medical device while exposing the adhesive surface of the device and applying the device to the skin of the user at a selected adhesion site on the user's body.

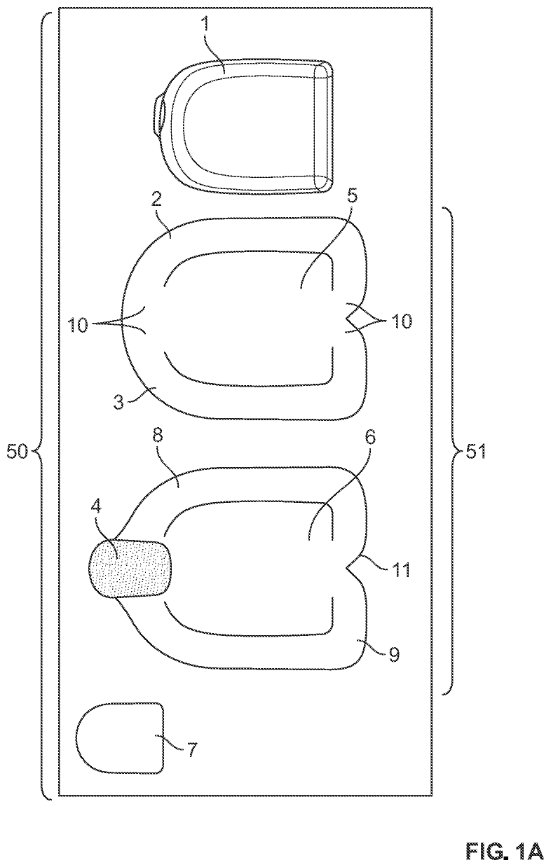

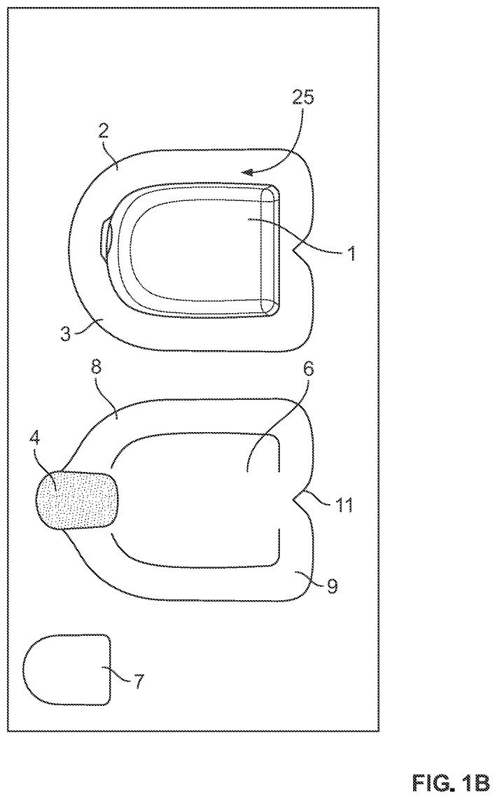

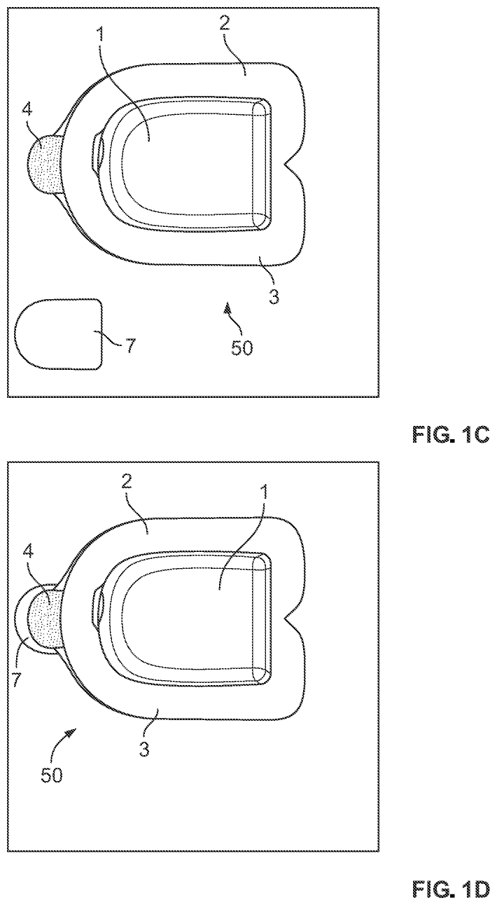

FIGS. 1A-1D illustrate an exemplary embodiment of an adhesive wearable drug delivery system 50 for delivering medication to a user. In some embodiments, the adhesive wearable drug delivery system 50 includes a drug delivery device 1, an adhesive platform 5, a temporary backing 6, and a needle cap 7. In some examples, the adhesive wearable drug delivery system 50 may be configured in four parts.

In some embodiments, a drug delivery device 1 includes a cannula and/or a needle, a needle actuator, a medication reservoir, optionally, a sensor, such as one or more of a motion sensor, an analyte sensor, a medical condition sensor, or an environmental sensor, and a processor, and/or other elements, components, or devices as may be described herein. The drug delivery device 1 may be configured to couple with the needle cap 7. Additional details of an example of a drug delivery device may be found in U.S. Pat. No. 10,716,896, the entire disclosure of which is incorporated herein by reference.

FIG. 1A illustrates an embodiment of an adhesive wearable drug delivery system. In FIG. 1A, the adhesive wearable drug delivery system 50 includes an adhesive platform device 51. The adhesive platform device 51 includes an adhesive platform 5 and a temporary backing 6. The adhesive platform device 51 may have a first bridge 2 and a second bridge 3 extending, respectively, from a first side of the adhesive platform 5 and a second side of adhesive platform 5 side. In some examples, there may be a gap or window between each bridge 2 and 3 and the adhesive platform 5. In some embodiments, the temporary backing 6 has a first wing 8 and a second wing 9 extending from a first side and an opposite second side of the temporary backing 6. In some embodiments, there is a gap or window between wings 8 and 9 and the temporary backing 6 that may match the gap or window of the adhesive platform 5. In some embodiments, the temporary backing 6 includes a temporary backing tab 4 extending from an edge of the temporary backing 6.

The temporary backing 6 has, for example, a notch 11 disposed in the body of the temporary backing 6 as shown in FIGS. 1A and 1B. The notch 11 may be disposed between temporary backing wing 8 and temporary backing wing 9. In some embodiments, the notch 11 may be tapered towards the center of the temporary backing body 6. The notch 11 may create a stress concentration to aid the peeling of the temporary backing 6 in one direction followed by the peeling of the temporary backing wing 8 and temporary backing wing 9 in an opposite direction to remove the entire temporary backing 6 as discussed herein. In an example, a temporary backing tab 4 may be disposed between the first temporary backing wing 8 and the second temporary backing wing 9 as shown in FIGS. 1A. In an example, the temporary backing 6, the temporary backing wing 8, and the temporary backing wing 9 are all one component. The temporary backing wing 8 and the temporary backing wing 9 may each extend from the temporary backing 6.

FIG. 1B illustrates a view with the drug delivery device 1 attached to the adhesive platform 5 of FIG. 1A. The adhesive platform 5 is beneath the drug delivery device 1 in this view. The adhesive platform 5 may be configured with adhesive, one or more heat stakes, or combinations thereof that are operable to couple to a bottom surface (i.e., the surface interfacing with the patient or user) of the drug delivery device 1. The adhesive platform 5 may be configured to facilitate attachment of the drug delivery device 1 to a patient's skin when the temporary backing 6 is removed. In some examples, the adhesive platform 5 may be made of one or more flexible materials such as gauze, fabric, rubber, latex, or any other material, which may be arranged in one or more layers. In addition, an adhesive substance may be disposed on at least a portion of a patient-facing surface of the adhesive platform 5 that is covered by a temporary backing 6. In other embodiments, an adhesive substance is disposed on at least a portion of each side (i.e., the first platform surface 25 and the second platform surface 26) of the adhesive platform 5. For example, an adhesive substance may be disposed on the second platform surface 26 of the adhesive platform 5 that interfaces with the drug delivery device 1. In some embodiments, an adhesive substance is disposed on the entirety of the patient-facing side of the adhesive platform 5 that contacts the skin of the patient and that is protected by the temporary backing 6. In some examples, the adhesive platform 5 extends radially outward beyond a perimeter of the drug delivery device 1 to provide a tolerance for placement of the drug delivery device 1 on the adhesive platform 5.

As illustrated in FIGS. 1C and ID, for example, the drug delivery device 1 may be attached on one side to the and on the other side to a temporary backing 6 adhesive platform 5. The temporary backing 6 may be secured to the second platform surface 26 of the adhesive platform 5 with adhesive, heat stakes, combinations thereof, or the like. In some examples, the temporary backing 6 may be made of one or more flexible materials such as paper, wax paper, mylar, or any other flexible material, which may be arranged in one or more layers. In addition, FIG. 1C shows the needle cap 7 removed. As can be seen in FIG. 1C, the needle cap 7 may be larger than the temporary backing tab 4, while in other examples the needle cap 7 may be smaller than the temporary backing tab 4. As shown in FIG. 1D, the temporary backing 4 may be positioned between a bottom surface of the drug delivery device 1 and the needle cap 7. Other arrangements of the needle cap 7 and temporary backing 4 may be provided.

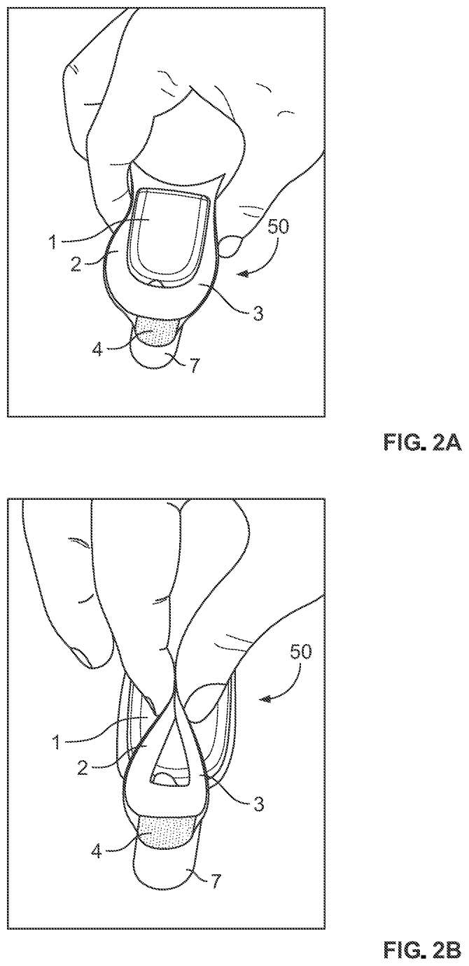

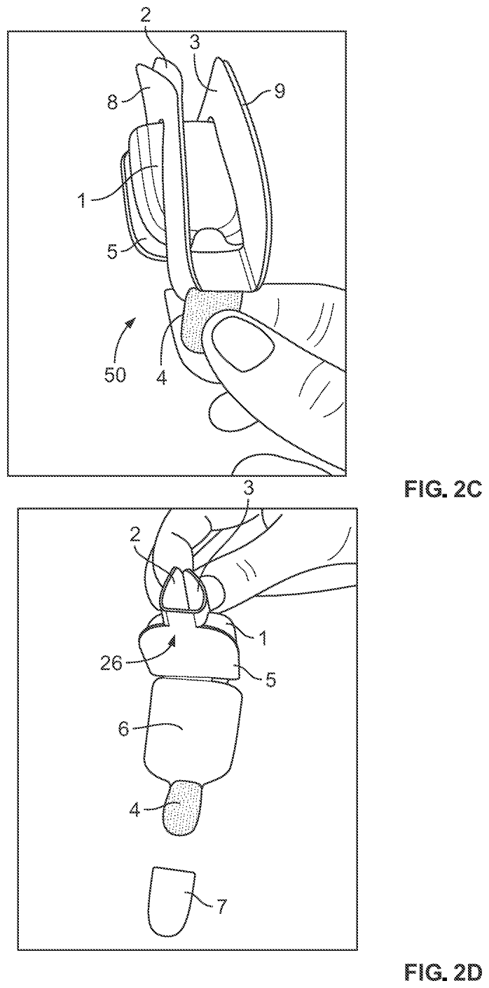

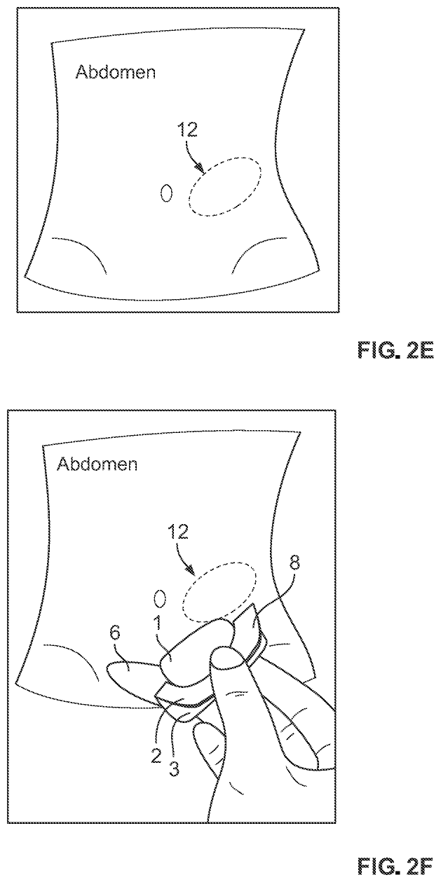

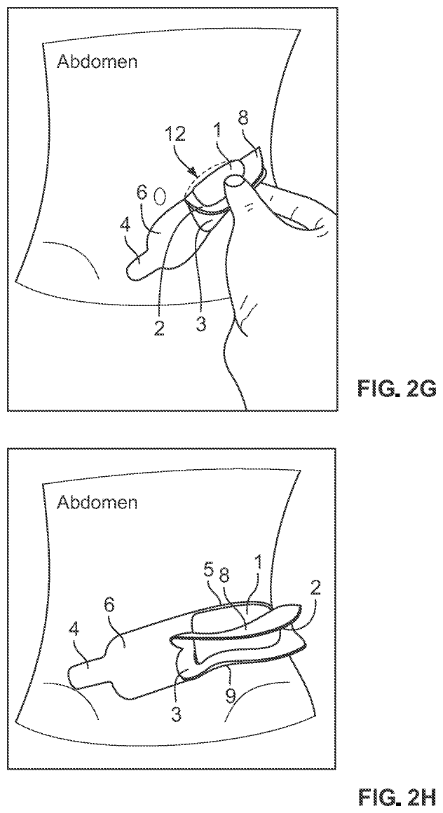

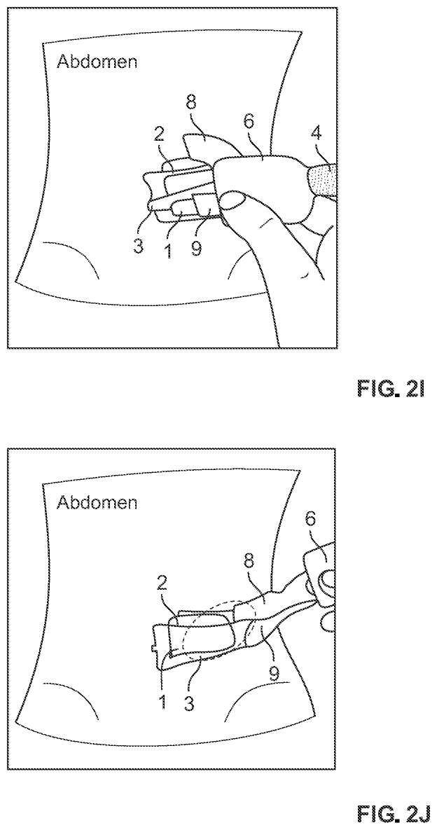

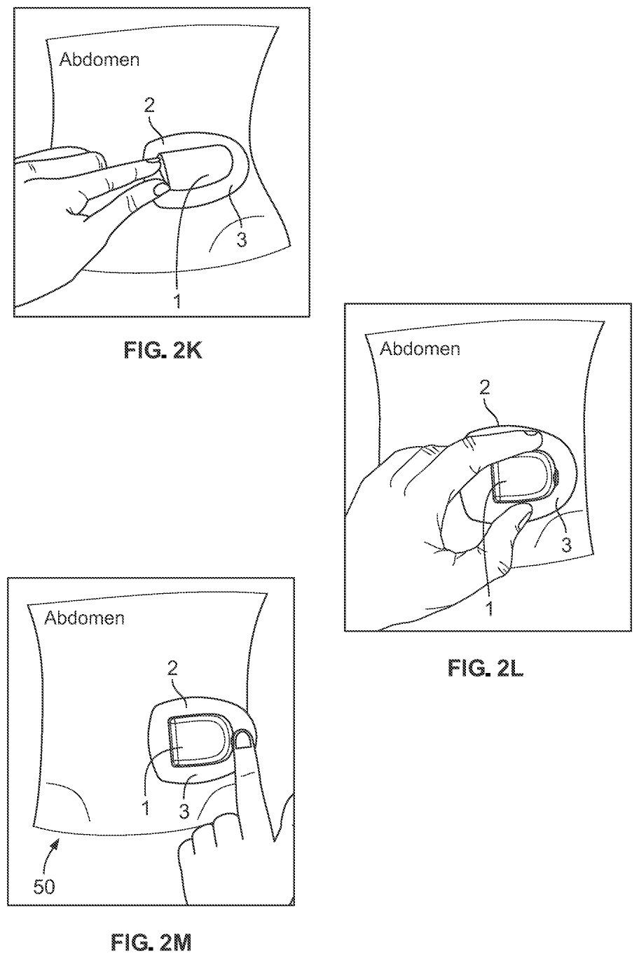

FIGS. 2A-2M illustrate various views of the first exemplary embodiment of the drug delivery device application for use with the wearable drug delivery device and an exemplary method of application. As shown in FIG. 2A, for example, the adhesive platform bridges 2 and 3 may be configured to rotate upwards enabling the adhesive platform bridges 2 and 3 to be used as handles of the drug delivery device 1. In some embodiments, the adhesive platform bridges 2 and 3 may be configured to couple together or at least touch one another when they are pulled up over the drug delivery device 1 as shown in FIG. 2B.

In the example illustrated in FIG. 2C, the first wing 8 may be configured to pair and match in size and shape with the adhesive platform bridge 2 and the second wing 9 is configured to mate with and match in size and shape with the second adhesive platform bridge 3. The temporary backing 6 and wings 8 and 9 may be configured to be lifted by the user in an upward direction (with respect to the drug delivery device 1) towards a top portion of the drug delivery device 1. In some embodiments, the wings 8 and 9 are configured to pair together above the drug delivery device 1 enabling the drug delivery device 1 to be lifted and applied to the skin of the patient.

The medical device application system 50 may be designed to be used to apply an on-body medical device to a patient. The on-body medical device may be affixed with an adhesive platform 5. The adhesive platform 5 may partially surround a surface of the drug delivery device 1. The adhesive platform 5 may serve as the primary or a supporting mechanism to affix the drug delivery device 1 to a patient. For example, a drug delivery device facing surface of the adhesive platform 5 may be secured to the on-body medical system 50 through an adhesive disposed on the surface or through heat staking and a bottom surface of the adhesive platform 5 may secure the drug delivery device 1 to the skin of a user at an adhesion site 12.

It may be beneficial to describe a process for applying a medical device using the adhesive drug delivery application device. While the figures are described herein with reference to a drug delivery device, the described adhesive drug delivery application device may be utilized with any medical device operable to be applied to the skin of the user or with a medical device (e.g., a sensor or the like) that may be modifiable to function when applied to the skin of a user.

FIGS. 2A-2M illustrate various views of the adhesive wearable drug delivery system 51 and steps of a method for applying the adhesive wearable drug delivery system 51 to an adhesion site 12.

In FIG. 2A, a user of the wearable system 50 may grasp the device by lifting an underside of the temporary backing wing 8 and the temporary backing wing 9. In some embodiments, the adhesive platform bridge 2 may be mated with the temporary backing wing 8 and the adhesive platform bridge 3 may be mated with the temporary backing wing 9. Therefore, when a user lifts the underside of the temporary backing wing 8 and the temporary backing wing 9 may also be lifting the adhesive platform bridge 2 and the adhesive platform bridge 3. The mated wings and bridges may be lifted to create a temporary handle above the drug delivery device 1 as shown in FIG. 2B. The needle cap 7 may be removed. After removal of the needle cap 7, the temporary backing 6 affixed to an adhesive platform 5 may be removed by pulling on a temporary backing tab 4, which causes the temporary backing 6 to pull away from the adhesive platform 5 as shown in FIGS. 2C and 2D. The wearable adhesive drug delivery system 51 is applied to an adhesion site 12 of the patient by pressing it to the patient's skin as shown in FIGS. 2F and 2G. The adhesion site may be, for example, a thigh, an arm, an abdomen, or the like of a patient or user.

Once the adhesive backing 5 has adhered to the adhesion site 12, the temporary handle may be released on the first temporary backing wing 8 and the second temporary backing wing 9. The drug delivery device 1 and the adhesive platform 5 remains attached to adhesion site 12 as shown in FIG. 2H. The temporary backing first wing 8 and temporary backing second wing 9 may be removed by pulling the temporary backing 6 in a direction opposite the direction that the temporary backing 6 to remove from the adhesive platform 5 as shown in FIGS. 21 and 2J. FIG. 21 illustrates an example of the first temporary backing wing 8 and the second temporary backing wing 9 approximately 50% removed from the respective first adhesive platform bridge 2 and the second adhesive platform bridge 3. FIG. 2J illustrates an example of the first temporary backing wing 8 and the second temporary backing wing 9 approximately 90% removed from the respective first adhesive platform bridge 2 and the second adhesive platform bridge 3. The temporary backing wings 8 and 9 may also be removed by pulling the temporary backing body 6 up above drug delivery device 1 and away from the adhesive platform bridges 2 and 3. The notch 11 may aid in the change of direction of peeling the temporary backing 6 off by creating a stress concentration. In some embodiments, removing the temporary backing first bridge 8 and the temporary backing second bridge 9 may result in the adhesive 10 of the underside of the adhesive platform first bridge 2 and adhesive platform second bridge 3 to be exposed. The adhesive platform first bridge 2 and adhesive platform second bridge 3 may be affixed by applying downward pressure on the drug delivery device facing side of the bridges as shown in FIGS. 2K-2M. In some embodiments, affixing the drug delivery device is configured to affix to the patient's skin by pressure applied first to the portion of the adhesive platform 5 closest to the notch 11 (see e.g., FIG. 2K), followed by the pressure applied to the middle of the adhesive platform bridges (see e.g., FIG. 2L), and to the portion of the adhesive platform 5 (see e.g., FIG. 2M) that was initially adjacent to the temporary backing tab 4.

The other embodiments described herein may be similarly applied with variations or modifications (including additional or deleted steps) made to the above described process according to the other embodiments respective configurations.

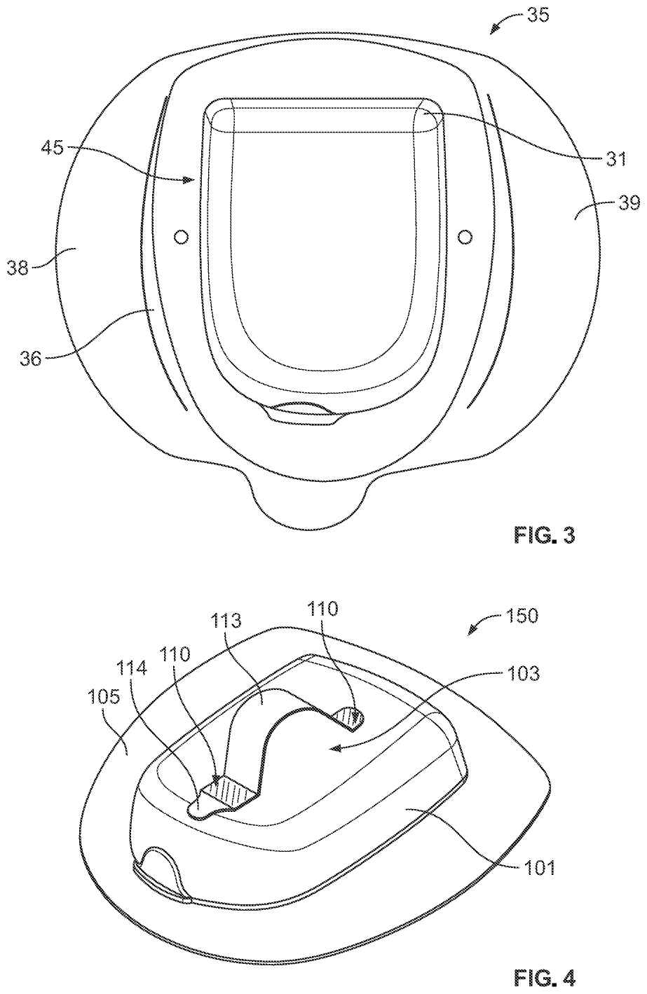

FIG. 3 illustrates a second exemplary embodiment of the drug delivery application device. In FIG. 3, for example, an adhesive wearable drug delivery system 35 may include a temporary backing 36, a temporary backing tab 34, a first temporary backing wing 38, and a second temporary backing wing 39 that may be configured as a single component. Alternatively, the adhesive wearable drug delivery system 35 may be assembled from separate components where the separate components may include a temporary backing tab 34, a temporary backing 36, a first temporary backing wing 38, and a second temporary backing wing 39 that are configured to couple together. In some embodiments, an adhesive platform 45 extends radially from the edge of the drug delivery device 31 to form a margin (i.e., the area between the outer edge of the drug delivery device. In some embodiments, the margin may taper in thickness as the margin extends away from the drug delivery device 31.

FIG. 4 illustrates an example of a medical device application system 150 including a temporary loop 113. As shown in FIG. 4, for example, the medical device application system 150 includes a drug delivery device 101, an adhesive platform 105, the temporary loop 113, and a temporary loop tab 114. In an example, the temporary loop 113 is attached to the external facing surface 103 of the drug delivery device 101. The temporary loop 113 may be attached to the drug delivery device 101 with adhesive 110 disposed between the concave surface of the temporary loop 113 and the external facing surface 103 of the drug delivery device 101. In some embodiments, the temporary loop 113 may have a temporary loop tab 114 extending from at least one of the adhered ends of the temporary loop 113. The adhesive 110 used to attach the temporary loop 113 to the external facing surface 103 of the drug delivery device may, for example, be configured to allow the temporary loop 113 to be detached from and reattached to the drug delivery device 101. The temporary loop 113 may be detached from the external facing surface of drug delivery device 101 by pulling the temporary loop 113 in a direction perpendicular to the external facing surface 103 of the drug delivery device 101, pulling the temporary loop tab 114, or a combination thereof. For example, two or more arches 113 are attached to the externally facing surface of the drug delivery device 101. The temporary loop 113 may be configured to allow one or more human fingers to pass between the concave surface of the temporary loop 113 and the external facing surface 103 of the drug delivery device 101. The user of the medical device application system 150 may lift the medical device application system 150 by sliding one or more fingers between the concave surface of the temporary loop 113 and the external facing surface 103 of the drug delivery device 101 thereby exposing the underside of the adhesive platform 105 for attachment to the skin of the patient at an adhesion site 12 (as shown in FIGS. 2E-F). In an example, the temporary backing 105 extends away from the drug delivery device 101. In some embodiments, the adhesive platform 105 is tapered at one end with a narrow end of the taper furthest from the drug delivery device 101.

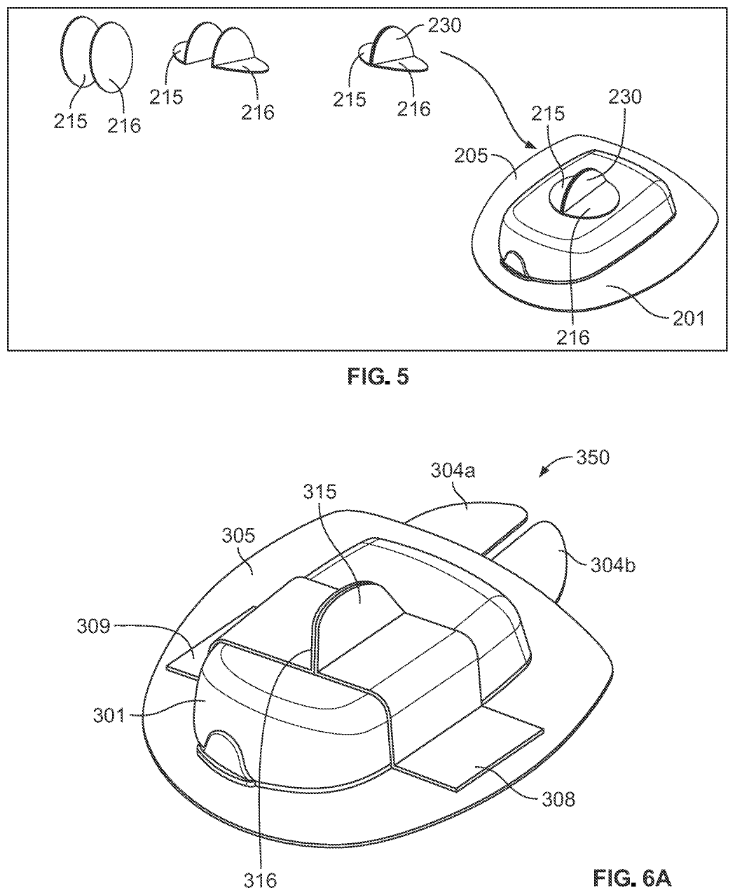

FIG. 5 illustrates an example of a wearable drug delivery device 201 and an exemplary method of assembly of a medical device application system 250 utilizing a temporary knob or grip. As shown in FIG. 5, some embodiments of the medical device application system 250 have a first sticker 215 and a second sticker 216 which are at least partially attached to the external facing surface of the drug delivery device 201 and an adhesive platform 505 attached to the internal facing surface (not shown) of the drug delivery device 201. The shape of the stickers may be substantially circular, oval, square, rectangular, trapezoidal, rhomboid, or any other shape that allows a handle to be formed. In some embodiments, stickers 215 and 216 are symmetrical. In some embodiments, stickers 215 and 216 are the same shape.

The stickers 215 and 216 may be assembled in combination with the drug delivery device 201 by bending or folding at least a portion of the sticker 215 and 216 and adhering a portion of sticker 215 to a portion of sticker 216 as shown in FIG. 5. The portion of sticker 215 adhered to the portion of sticker 216 forms a grip ridge or knob 230. The portions of sticker 215 and sticker 216 not adhered to each other (i.e., the portions that do not form the knob 230) may be adhered to the external facing surface of the drug delivery device 201. The knob 230 may be used to lift the medical device application system 250 exposing the adhesive platform 205 and allow a user of the medical device application system 250 to apply the device to the skin at an adhesion site. The stickers 215 and 216 may be configured to be detachably connected to an external surface of drug delivery device 201 with an adhesive configured to permit removal of the stickers by force or peeling the stickers 215 and 216 off the external facing surface of the drug delivery device 201. The user may remove sticker 215 and 216 after the medical device application system 250 is applied to the skin of a patient. In some embodiments, one sticker, such as 215, may be used to create a grip or knob on the external facing surface of the drug delivery device 201. In other embodiments, three or more stickers 215 is used to create a grip or knob, such as for example, 230, on the external facing surface of the drug delivery device 201.

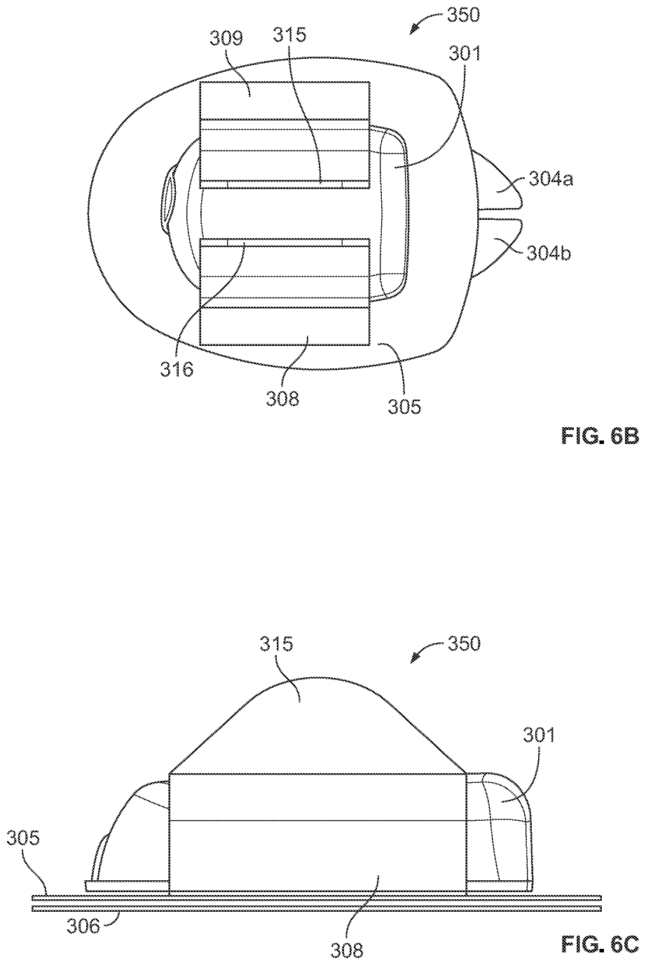

FIGS. 6A-6C illustrate, for example, an embodiment of a medical device application system 350 including a drug delivery device 301, a temporary backing first tab 304a, a temporary backing second tab 304b, an adhesive platform 305, a temporary backing 306, a first temporary wing 308, a second temporary wing 309, first ridge 315, and a second ridge 316. FIG. 6A illustrates a top-down angled view of the medical device application system 350. FIG. 6B illustrates a top-down view of FIG. 6A. FIC. 6C illustrates a side view of FIG. 6A. In some embodiments of FIGS. 6A-6C, the drug delivery device 301 is the same as in earlier example, but the adhesive platform 305 may differ from earlier examples. For example, the temporary backing of first wing 308 and second wing 309 may be disconnected from the temporary backing 306 and attached to the external facing surface of the adhesive platform 305. The attachment location of the temporary wings 308 and 309 on the adhesive platform 305 may be on respective side of the drug delivery device 301 as shown in FIGS. 6A-6C. In some embodiments, the attachment mechanism used to attach temporary wing 308 to the adhesive platform 305 is an adhesive, heat stakes, or combinations thereof. In an example, the attachment mechanism used to attach the temporary wings 308 and 309 to the adhesive platform 305 may be a weak adhesive to allow the temporary wings 308 and 309 to be removed by applying a torsional force away from the surface of the adhesive platform 305 or peeling the temporary wings 308 and 309 off the adhesive platform 306, or combinations thereof.

In some embodiments, the temporary backing first wing 308 and second wing 309 are disconnected from the temporary backing body 306 and attached to the medical device application system 350 between the drug delivery device 301 and the temporary backing wings 308 and 309 extend outward towards the drug delivery device 301.

In some embodiments, the temporary backing first wing 308 and temporary backing second wing 309 extend from an area adjacent to or beneath the internal facing surface of the drug delivery device 301 above the drug delivery device 301 to form a first ridge 315 and a second ridge 316. In some embodiments, the temporary backing first wing 308 has excess length that extends beyond the drug delivery device 301 to form a first ridge 315 as shown, for example, in FIG. 6A.

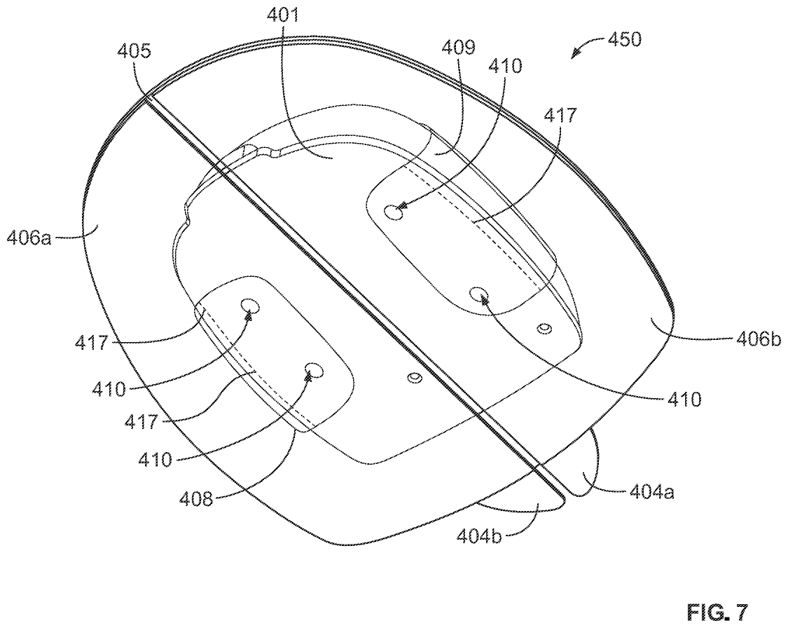

FIG. 7 illustrates a patient facing isometric view of an exemplary embodiment of a medical device application system 450 including a drug delivery device 401, a temporary backing first tab 404a, a temporary backing second tab 404b, an adhesive platform 405, a first temporary backing component 406a, a first temporary backing component 406b, a first temporary wing 408, a second temporary wing 409, heat stakes 410, and perforated edges 417. In an example, the first and second temporary backing wings 408 and 409 are attached to the skin facing surface of the drug delivery device 401 using one or more heat stakes 410. The medical device application system 450 may have one or more perforated edges 417 disposed through the first wing 408 and second wing 409. In some embodiments, the perforated edges are configured to align with the outer edge of the drug delivery device 401. In some embodiments, one or more perforated edges 417 are configured to permit the first temporary backing wing 408 and/or second temporary backing wing 409 to be removed by tearing the respective temporary backing wings 408 and 409 along the respective perforated edges 417. In some embodiments, the temporary backing wings 408 and 409 are configured to be detached and removed from the medical device application system 450 by tearing along the perforated edges 417 after the medical device application system 450 has been adhered to the skin of a patient.

In some embodiments, the temporary backing first wing 408 and temporary backing second wing 409 are both attached to a patient facing internal surface of the drug delivery device 401 and do not overlap as shown in FIG. 7. In some embodiments, the detached wings 408 and 409 are attached to the underside of the drug delivery device 401 with adhesive 410, heat stakes, or combinations thereof. In some embodiments, temporary backing first wing 408 and temporary backing second wing 409 have perforated edges 417 configured to remove a portion of the temporary backing wings 408 and 409 after the medical device application system 450 has been applied to the patient by tearing the temporary backing first wing 408 and temporary backing second wing 409 at the perforated edges 417. In some embodiments, the perforated edge 417 is disposed through the temporary backing wings 408 and 409 where the edge of the drug delivery device 401 meets the adhesive platform 405.

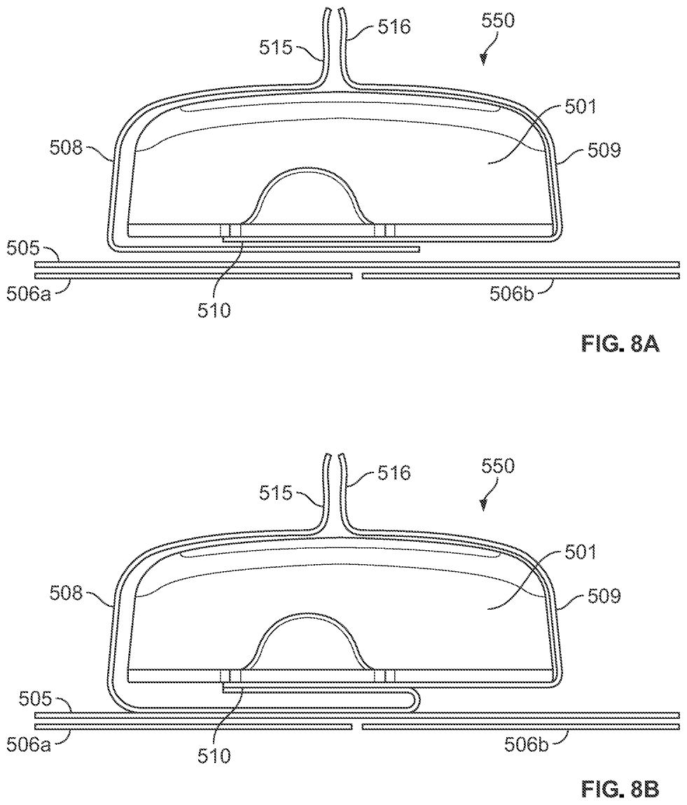

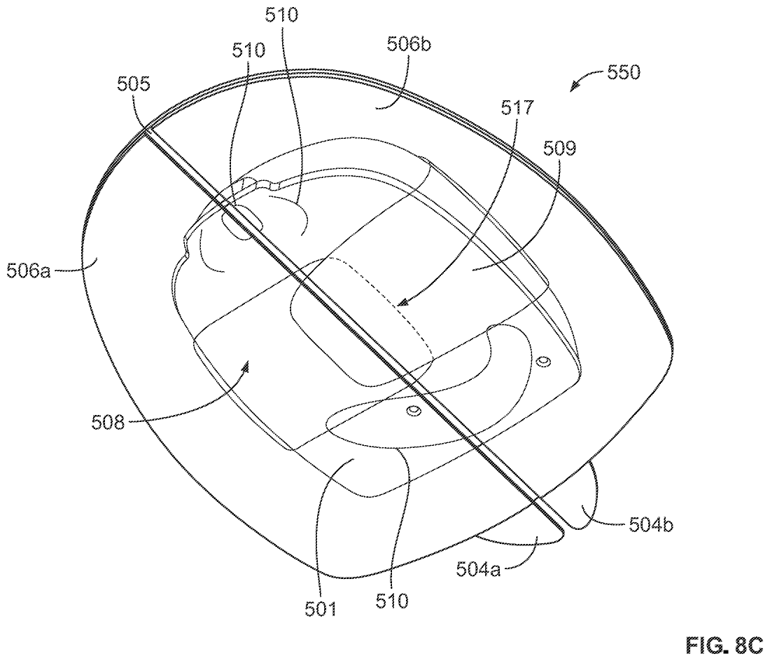

FIGS. 8A-8C illustrate views of an example of a medical device application system 550 including temporary wings extending from an internal facing surface of a drug delivery device 501. FIG. 8A illustrates an example of the medical device application system 550 having a drug delivery device 501, an adhesive platform 505, a first temporary backing component 506a, a first temporary backing component 506b, a temporary backing first wing 508, a temporary backing first wing 509, a first ridge 515, and a second ridge 516.

In an example, the adhesive drug delivery application device 550 may include a temporary backing second wing 509 that may have excess length that extends beyond the drug delivery device 501 to form a second ridge 516. A user of the adhesive wearable drug delivery application device 550 may pinch or grip the first ridge 515 and second ridge 516 together to lift or hold the drug delivery device 501 and expose the internally facing surface of adhesive platform 505 to apply the adhesive wearable drug delivery application device 550 to the skin of the patient at an adhesion site.

FIG. 8B illustrates 8A with the temporary backing first wing 508 uncompressed under the drug delivery device 501. In some embodiments, the temporary backing second wing 509 may be configured to have an adhesive 510 that is disposed on an end of the internal facing surface to facilitate mating of the temporary backing first wing 508 and temporary backing second wing 509 between the drug delivery device 501 and adhesive platform 505. In an example, the adhesive 510 is disposed on the end of the internal facing side of the temporary backing second wing 509 as shown in FIG. 8B, the temporary backing second wing 509 may be configured to fold over beyond an area where it mates with the temporary backing first wing 508 to extend away from temporary backing first wing 508 and towards drug delivery device 501. In an example, a portion of the second temporary backing wing 509 may extend above the drug delivery device 501 to form a first ridge portion 515. In addition, a portion of the first temporary backing wing 509 may extend above the drug delivery device 501 to form a second ridge portion 516. In some embodiments, the first ridge and the second ridge pair to form a handle.

As illustrated in FIG. 8C, for example, the temporary backing 506 may comprise at least a first component 506a and a second component 506b. In some embodiments, a first and second components of temporary backing 506a and 506b each have a tab 504a and 504b extending from the edge of the body. The temporary backing tab 504, tabs 504a and 504b, or the tapered section of the temporary backing 506 may be gripped by the user of the medical device application system 550 to peel or rip the temporary backing away 506 from the adhesive platform 505.

Further referencing FIG. 8C, the temporary backing first wing 508 and temporary backing second wing 509 are attached to the patient facing internal surface of the drug delivery device 501. In some embodiments, the temporary backing first wing 508 is configured to have adhesive 510 disposed on the end of the internal facing surface to facilitate mating between the temporary backing first wing 508 and the temporary backing second wing 509 between the internal facing surface of the drug delivery device 501 and the external facing surface of the adhesive platform 505. The internal facing surface of the drug delivery device may be the surface of the drug delivery device facing the skin of the patient. In an example, the temporary backing second wing 509 may at least partially overlap the temporary backing first wing 508. In some embodiments, the temporary backing second wing 509 may have a perforated edge 517 at least partially disposed along the latitudinal axis. In some embodiments, the temporary backing second wing 509 may have a perforated edge 517 disposed through the entirety of the latitudinal axis of the temporary backing second wing 509. The perforated edge 517 may align with the edge of the temporary backing first wing 508. In some embodiments, the perforated edge 517 is configured to be torn along the perforated edge 517, allowing at least one of the temporary backing first wing 508 and the temporary backing second wing 509 to be removed from the medical device application system 550.

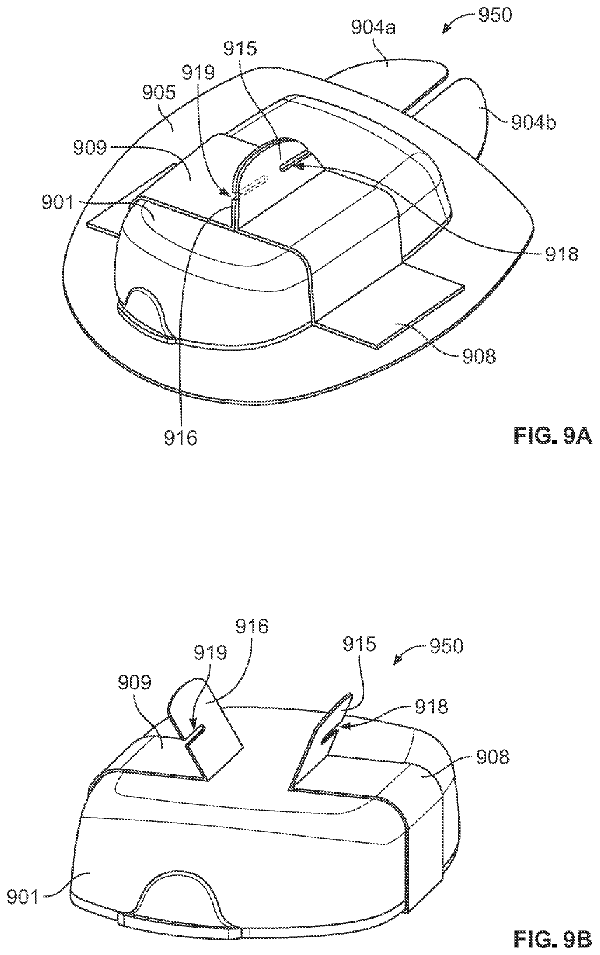

FIGS. 9A and 9B illustrate perspective views of a medical device application system 950 with a locking slot mechanism. In FIGS. 9A and 9B, an example of a medical device application system 950 including a drug delivery device 901, a temporary backing first tab 904a, a temporary backing second tab 904b, an adhesive platform 905, a first temporary wing 908, a second temporary wing 909, a first ridge portion 915, a second ridge portion 916, a first slot 918, and a second slot 919.

In an example, the temporary backing first wing 908 or first temporary backing ridge 915 has a first slot 918 at least partially disposed therein. In some embodiments, the temporary backing second wing 909 or first temporary backing ridge 916 may have a second slot 919 at least partially disposed therein as shown in FIG. 9B. In some embodiments, the first slot 918 is disposed through the ridge 916 at the opposite direction as second slot 919 is disposed through the ridge 916. First slot 918 may be configured to interface with the second slot 919 to lock together the ridges 915 and 916 and form a locked grip or handle.

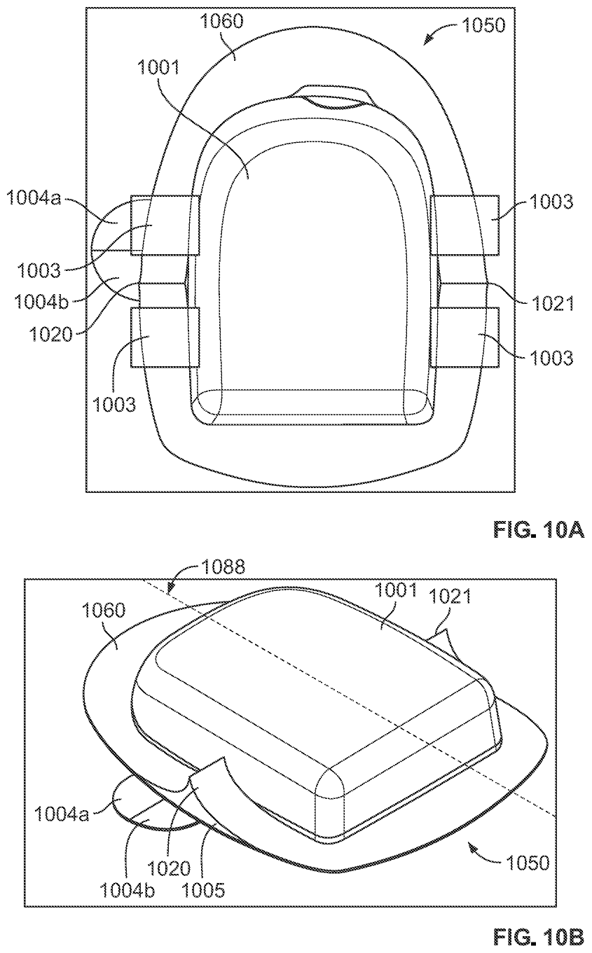

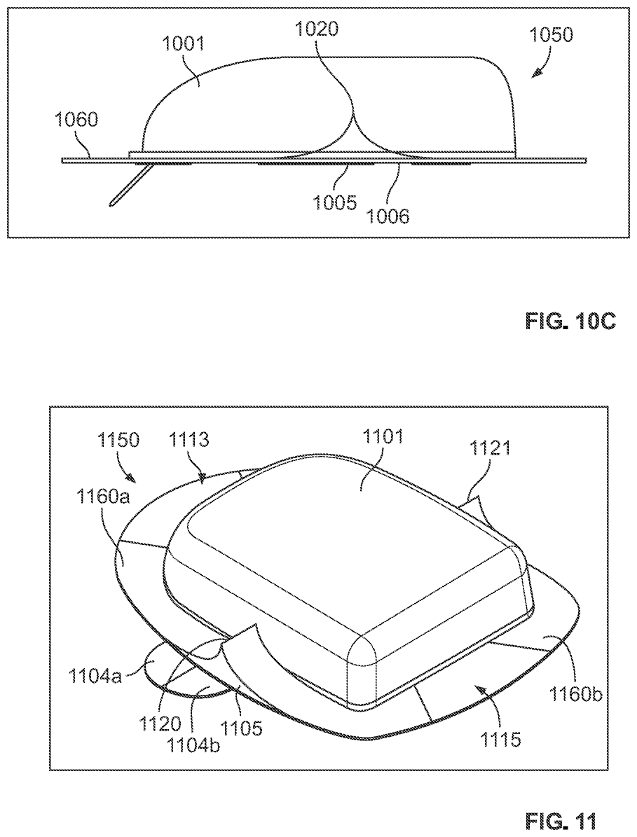

FIGS. 10A-C illustrate an example of a medical device application system 1050 with a

moveable layer 1060. FIG. 10A illustrates a top-down view of an example of a medical device application system 1050. FIG. 10B illustrates a two point top-side-angled view of an example of a medical device application system 1050. FIG. 10C illustrates a side view of an example of a medical device application system 1050. The medical device application system 1050 example illustrated in FIG. A includes a drug delivery device 1001, adhesive 1003, a temporary backing first tab 1004a, a temporary backing second tab 1004b, a first temporary handle 1020, a second temporary handle 1021, and the moveable layer 1060.

As shown in FIGS. 10A-10C, the adhesive platform 1005 (shown in FIG. 10B) may have a moveable layer 1060 which interfaces with an external facing surface of the drug delivery device 1001 where the adhesive platform 1005 extends from the drug delivery device 1001.

The moveable layer 1060 may be movable relative to the adhesive platform 1005 to allow it to be pinched and lifted by the user of the medical device application system 1050. In some embodiments, the moveable layer 1060 may cover the entire region between an edge of the drug delivery device 1001 and an outer edge of the adhesive platform 1005 as shown, for example, in FIG. 10A. The moveable layer 1060 may be attached to the adhesive platform 1005 with adhesive 1003 disposed at least partially on the internal facing side of the moveable layer 1060. The moveable layer 1060 may be attached to the adhesive platform 1005 with one or more heat stakes. The moveable layer may, in some examples, have up to four or more attachment points 1003 as shown with squares in FIG. 10A to the adhesive platform 1005 that are spaced adjacent to the temporary handles 1020 and 1021. In some embodiments, as shown in at least FIG. 10B, the adhesive platform 1005 extends radially outwards from drug delivery device 1001 to form a margin area between the outer edge of the drug delivery device 1001 and the outer edge of the adhesive platform 1005. As used herein, the term “margin” may refer to a border, a boundary, or a perforated section.

As shown in FIG. 10B, the moveable layer 1060 may be configured to have a first temporary handle 1020 and a second temporary handle 1021. The moveable layer 1060 may be movable relative to the adhesive platform 1005 to allow the user of the adhesive wearable drug delivery application device 1050 to lift at least a portion of the moveable layer 1060 to form a first temporary handle 1020 and a second temporary handle 1021.

The temporary handles 1020 and 1021 may be configured to lie flat against the adhesive platform 1005 or itself or a combination thereof after the adhesive wearable drug delivery application device 1050 has been applied to the skin of the patient at the adhesion site. For example, the temporary handles 1020 and 1021 may lie flat naturally when the moveable layer 1060 is not pinched by the user. The temporary handles 1020 and 1021 may, for example, have an adhesive disposed on the internal facing side of the movable layer 1060 enabling the temporary handles 1020 and 1021 to lie flat against the adhesive platform 1005. Alternatively, or in addition, the adhesive platform 1005 may have an adhesive disposed at least partially onto the external facing surface to pair with the temporary handles 1020 and 1021 and allow the handles to lie flat. In the examples, a temporary backing layer 1006 may cover the adhesive used to mate with the temporary handles 1020 and 1021 prior to application of the medical device application system 1050 and is removed after application of the medical device application system 1050 to the skin of a user. In some embodiments, the temporary handles 1020 and 1021 are formed from moveable layer 1060 of material where the pinch axis is perpendicular to a longitudinal axis 1088 of the drug delivery device 1001 as illustrated, for example, in FIG. 10B. The movable layer 1060 may be formed from different materials that maybe flexible, semirigid, or a combination of both.

FIG. 11 illustrates an exemplary medical device application system a medical device application system 1150 with a drug delivery device 1101, a temporary backing first tab 1104a, a temporary backing second tab 1104b, an adhesive platform 1105, a first side 1113 and a second side 1115, a first temporary handle 1120, a second temporary handle 1121, a first movable layer component 1160a, and a second movable layer component 1160b. In an example, the moveable layer may comprise the first movable layer component 1160a having the first handle 1120 and the second movable layer component 1160b having the second handle 1121. In some embodiments, the movable layer first component may cover two sides, i.e., first side 1113 and second side 1115, respectively, of the adhesive platform 1105 and leave two sides of the adhesive platform 1105 exposed.

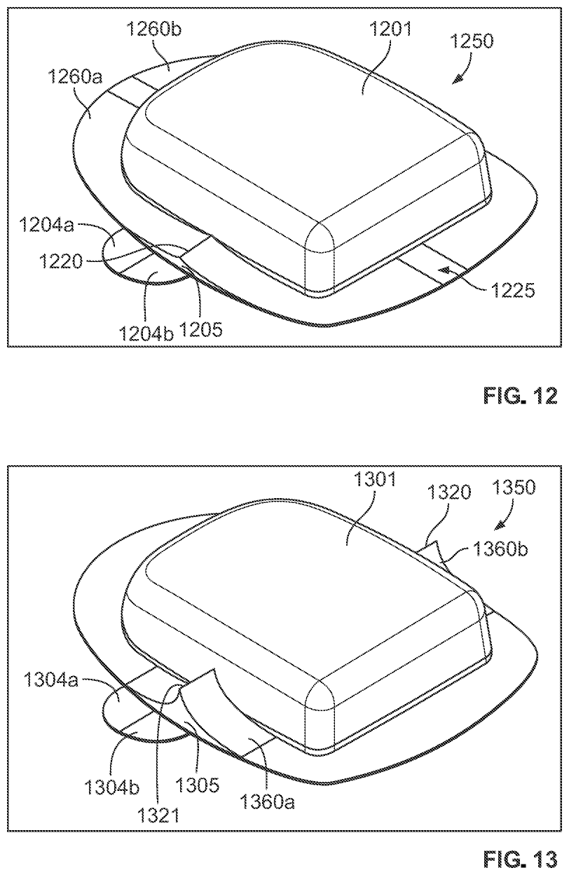

As shown in FIG. 12, for example, the drug delivery application device 1250 may include a movable layer first component 1260a may have a first temporary handle 1220 and cover about half the adhesive platform 1205 and another movable layer second component 1260b has a second temporary handle 1221 (not shown) and covers about half the adhesive platform 1205. In some embodiments, the movable layer first component 1260a and the movable layer second component 1260b each cover about half the adhesive platform 1205 leaving a small gap 1225 of exposed adhesive platform 1205 between the first movable layer component 1260a and the second movable layer component 1260b. The small gap 1225 may extend the length of the longitudinal axis of the adhesive platform 1205.

FIG. 13 illustrates an exemplary embodiment where a movable layer first component 1360a forms a first temporary handle 1321. In some embodiments, of the adhesive drug delivery application device 1350, the movable layer first component 1360a may have a minimal size usable, such as 0.25 inches, 0.5 inches, 1 inch, or the like, to form a temporary handle 1321 and a movable layer second component 1360b forming a second temporary handle 1320. The movable layer second component 1360b may be of similar size as the movable layer first component 1360a. In some embodiments, the movable layer components 1360a and 1360b are each adhered to the adhesive platform 1305 at their respective first ends and second ends.

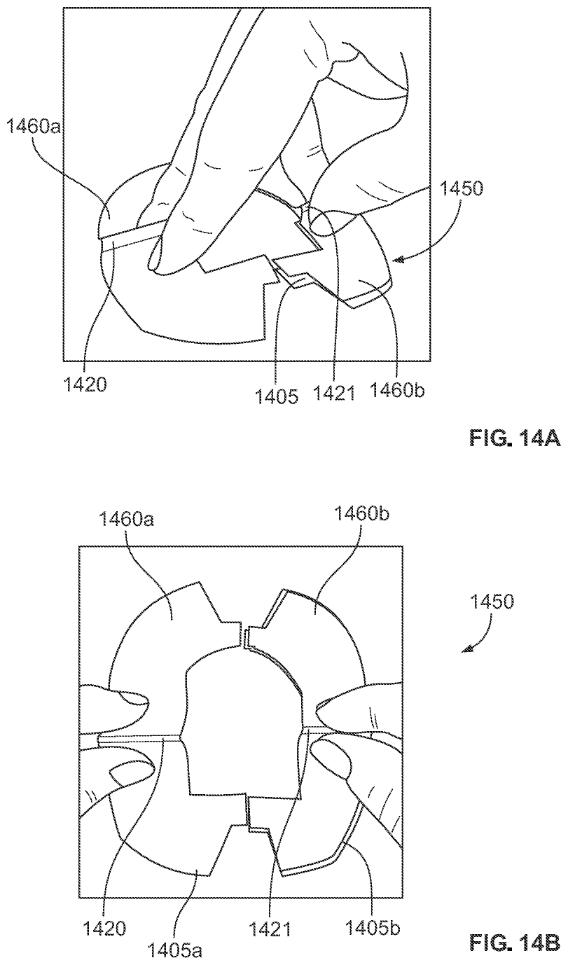

FIG. 14A shows an exemplary embodiment where a movable layer 1460 having a first component 1460a and a second component 1460b has a first temporary handle 1420 and a second temporary handle 1421 which may be pinched by the user of the medical device application system 1450 with one hand using at least two fingers on either side of the temporary handle. The one-handed grip on the temporary handles 1420 and 1421 may allow the user of the adhesive wearable drug delivery application device 1450 to lift the device and expose the adhesive 1410 on the back of the adhesive platform 1405a and 1405b and apply the device to the skin of the patient.

FIG. 14B shows an exemplary embodiment of the adhesive wearable drug delivery application device 1450. where a movable layer having a first component 1460a and a second component 1460b has a first temporary handle 1420 and a second temporary handle 1421 which may be pinched by the user of adhesive wearable drug delivery application device 1450 with two hands using at least two fingers on either side of the temporary handle. A two-handed grip on the temporary handles 1420 and 1421 may allow the user of the adhesive wearable drug application delivery device 1450 to lift a medical device (shown in other examples) and expose the adhesive shown or described with reference to other examples on the back of the adhesive platform 1405a and 1405b and apply the medical device using the adhesive wearable drug delivery application device 1450 to the skin of the patient.

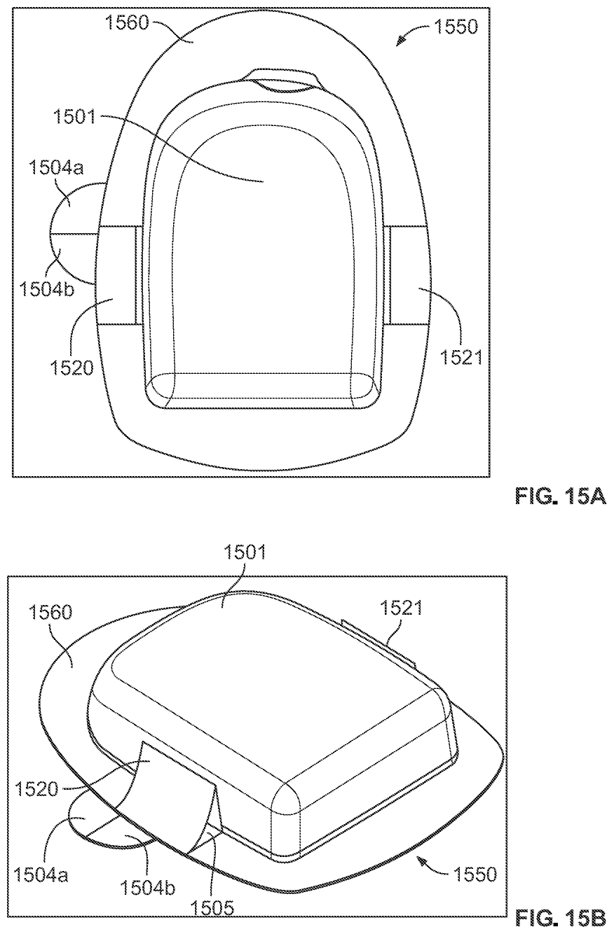

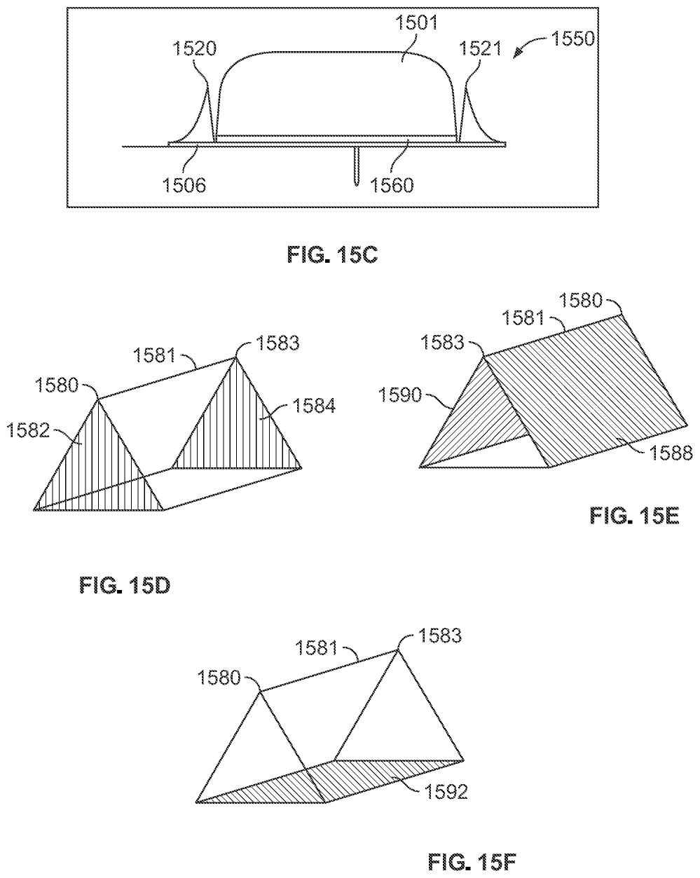

FIGS. 15A-C illustrate an exemplary embodiment of a medical device application system 1550 including one or more temporary handles 1520 and 1521 having a generally triangular prism shape. FIG. 15A illustrates a top-down view of an example of the medical device application system 1550. The medical device application system 1550 may include a drug delivery device 1501, a temporary backing first tab 1504a, a temporary backing second tab 1504b, a movable layer 1560, and an adhesive platform 1505 (see FIG. 15B). In some embodiments, one or more generally triangular prism shaped temporary handles 1520 and 1521 extend from an external facing surface of a moveable layer 1560 towards the drug delivery device 5101. FIG. 15B illustrates a two point angled view of 15A. FIG. 15C illustrates a side view of 15A.

FIGS. 15D-15F illustrate an exemplary embodiment temporary handle 1520 or 1521 having a generally triangular prism shape. FIG. 15D illustrates an exemplary embodiment of a temporary handle 1520 or 1521 with a first base 1582 and a second base 1584 shaded in grey. The temporary handle 1520 and/or the temporary handle 1521 may have a generally triangular prism shape may have a first vertex 1580 which is the furthest point of the first base 1582 extending away from the adhesive platform 1505. In addition, the temporary handles 1520 and 1521 may have a second vertex 1583 which is the furthest point of the second base 1584 extending away from the adhesive platform 1505. The temporary handles 1520 and 1521 may have a triangular prism edge 1581 extending from the first vertex 1580 to the second vertex 1583. In some embodiments, the triangular prism edge 1581 is oriented parallel to the longitudinal axis of the drug delivery device 1501. In other embodiments, the triangular prism edge 1581 may be oriented perpendicular to the longitudinal axis of the drug delivery device 1501. In some embodiments, the temporary handles 1520 and 1521 may have an opening extending from the first base 1582 to the second base 1584.

FIG. 15E illustrates an exemplary embodiment of a temporary handle 1520 or 1521 having a generally triangular prism shape which has a first lateral facing side 1588 and second lateral facing side 1590 shaded in grey. In some embodiments, at least one of the first and second lateral facing sides 1588 or 1590 have a concave surface curved inwards towards the center of the triangular prism. In an example, at least one of the first and second sides may have adhesive at least partially disposed on the surface. In an example, at least one of the first and second temporary handles 1520 and 1521 is configured to lie flat against itself. At least one lateral side 1588 or 1590 may be configured to move parallel to the adhesive platform 1505 to lie flat. Alternatively, or in addition, at least one lateral side 1588 or 1590 may be configured fold in half so each folded section and the other lateral face lies parallel to the adhesive platform 1505.

FIG. 15F illustrates an exemplary embodiment of a temporary handle 1520 or 1521 having a generally triangular prism shape which has an adhesive platform facing underside 1592 shaded in grey. In some embodiments, a first and second temporary handles 1520 or 1521 have an opening at least partially in the adhesive platform facing underside 1592 exposing the adhesive platform 1505.

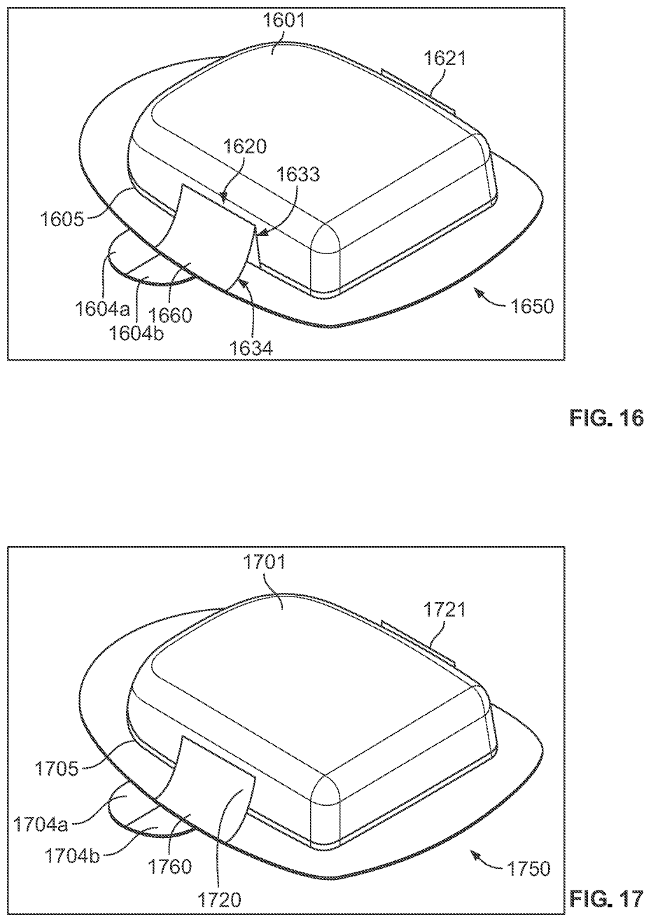

FIG. 16 illustrates an exemplary embodiment of a medical device application system 1650 where the movable layer 1660 forms a first temporary handle 1620 and a second temporary handle 1621. In an example, a first component of the movable layer 1660 may be the first temporary handle 1620, which forms a generally triangular prism shape. The first temporary handle 1620 may have a first lateral side 1633 at least partially affixed to the adhesive platform 1605 (closest to the drug delivery device 1601) and a second lateral side 1634 also at least partially affixed to the adhesive platform 1605. A second component of the movable layer 1660 is the second temporary handle 1621 (partially obscured by the drug delivery device 1601) that also forms a generally triangular prism shape having a first lateral side at least partially affixed to the adhesive platform 1605 and a second lateral side at least partially affixed to the adhesive platform 1605.

FIG. 17 illustrates an exemplary embodiment where the movable layer 1760 forms a first temporary handle 1720 and a second temporary handle 1721 (partially obscured by drug delivery device 1701). As illustrated in FIG. 17, for example, in some embodiments, the moveable layer 1760 may include two or more rectangular components which form a first temporary handle 1720 and a second temporary handle 1721. The first temporary handle 1720 and the second temporary handle 1721 may have an attachment mechanism, such as an adhesive only applied to where an outer edge of the moveable layer 1760 meets an outer edge of the adhesive platform 1705. In some embodiments, the movable layer 1760 may have a substantially circular, oval, half-circle, half oval, square, rhomboid, trapezoid, triangular, pentagonal, hexagonal, or any other shape capable of forming a temporary handle.

Referring now to FIG. 17, for example, there are numerous methods through which the medical device application system 1750 may be assembled. In some embodiments, the assembly of the medical device application system 1750 may comprise the following steps. Covering at least partially the internal facing side of an adhesive platform 1705 with an adhesive 1710. Placing temporary backing 1706 on top of the internal facing side of the adhesive platform 1705 coated with adhesive 1710. Cutting the combined temporary backing 1706 attached to the temporary backing 1706 to the desired shape. In some embodiments, the desired shape is the shape of the drug delivery device 1701 internal surface with additional surface extending beyond the drug delivery device 1701 radially. Disposing, at least partially, adhesive 1710 on the internal facing surface of the drug delivery device 1701, the external facing surface of the adhesive platform 1705, or combinations thereof. Attaching the drug delivery device 1701 to the external facing surface of the adhesive platform 1705. In some embodiments, heat staking 1710 is used instead of adhesive for attachment. In some embodiments, heat steaking is used in addition to adhesive 1710 for attachment.

In some embodiments, for example in reference to FIGS. 2A-2M, another method of assembly may include the following steps. In some embodiments, the method of assembly may include the step of adding the temporary backing wings 8 and 9. This embodiment may include covering a portion of the external facing side of the adhesive platform 5 with adhesive 10 configured to mate with the shape with the attachment portion of the temporary backing first wing 8 and the temporary backing second wing 9. Placing the temporary backing first wing 8 and temporary backing second wing 9 on the portions of adhesive platform 5 coated with adhesive 10. Attaching the drug delivery device 1 to the same the external facing surface of the adhesive platform 5. In some embodiments, the temporary backing 6 is cut into a shape having a main body and a temporary backing first wing 8 and a temporary backing second wing 9 extending from the body of the temporary backing 6. In some embodiments, the drug delivery device 1 is placed on the adhesive platform 5 prior to the temporary backing first wing 8 and the temporary backing second wing 9 being placed on the adhesive platform 5.

In some embodiments, for example in reference to FIGS. 2A-2M, another method of application may include the following steps. The method of applying a medical device application system 50 may include the step of moving the internal facing surface of the temporary backing first wing 8 and the temporary backing second wing 9 towards each other and away from the internal facing surface of the drug delivery device 1. The method of applying a medical device application system 50 may include the step of configuring the bridges 2 and 3 to touch above the external facing surface of the drug delivery device 1. The method of applying a medical device application system 50 may include the step of pinching the internal facing surfaces of the wings 8 and 9 to touch one another. The method of applying a medical device application system 50 may include the step of lifting the of the drug delivery device 1 by the temporary handle with the temporary backing first wing 8 and temporary backing second wing 9.

In some embodiments, for example in reference to FIGS. 2A-2M, another method for applying a medical device application system 50 is described. The method of applying a medical device application system 50 may include the step of holding the drug delivery device 1 with a temporary handle so the temporary backing first wing 8 and the temporary backing second wing 9, the temporary backing 6 and the temporary backing tab 4 may be exposed. The method of applying a medical device application system 50 may include the step of pulling the temporary backing 6 by the temporary backing tab 4 in a direction perpendicular to the patient facing surface of the drug delivery device 1 to remove the temporary backing 6. The method of applying a medical device application system 50 may include the step of pressing the patient facing side of the exposed adhesive platform to the adhesion site 12. In some embodiments, the method of applying a medical device application system 50 may include the step of applying a medical device application system 50 to the adhesion site 12 at the abdomen, thigh, arm, or any other area of skin suitable for delivering drug from a drug delivery device. The method of applying a medical device application system 50 may include the step of removing the remainder of the temporary backing 6, the temporary backing first wing 8, and the temporary backing second wing 9 by continuing to pull the backing away from the adhesive platform 5.

In some embodiments, such as, for example, FIGS. 10A-C the medical device application system 1050 is assembled including the addition of a moveable layer 1060. The steps for assembling the medical device application system 1050 with the moveable layer 1060 may include covering, at least partially, one side of the adhesive platform 1005 with an adhesive 1003 and then placing a temporary backing 1006 on the adhesive 1003. Some embodiments may include attaching the moveable layer 1060 to the adhesive platform 1005. In some embodiments, the step of attaching the temporary backing 1006 to the moveable layer 1060 includes configuring the location of adhesive to allow the formation of or include temporary handles 1020 and 1021 in the moveable layer 1060. Attaching the drug delivery device 1001 to the external facing surface of the adhesive platform 1005. In some embodiments, the method includes cutting the moveable layer 1060 into a shape at least partially covering the external facing surface of the adhesive platform 1005 not covered by the drug delivery device 1001 and attaching it to the adhesive platform 1005 before the drug delivery device 1001 is attached to the adhesive platform 1005. In some embodiments, the method includes cutting the moveable layer 1060 into a shape at least partially covering the external facing surface of the adhesive platform 1005 not covered by the drug delivery device 1001 and attaching it to the adhesive platform 1005 after the drug delivery device 1001 is attached to the adhesive platform 1005.

Each example of a drug delivery device as described herein may be interchangeable with drug delivery devices described in other examples or embodiments herein. For example, the drug delivery device 1701 described and illustrated in reference to FIG. 17, the drug delivery device 1601 described and illustrated in reference to FIG. 16, the drug delivery device 1501 described and illustrated in reference to FIGS. 15A-15F, the drug delivery device 1301 described and illustrated in reference to FIG. 13, the drug delivery device 1201 described and illustrated in reference to FIG. 12, the drug delivery device 1101 described and illustrated in reference to FIG. 11, the drug delivery device 1001 described and illustrated to FIGS. 10A-10C, the drug delivery device 901 described and illustrated in reference to FIGS. 9A-9B, the drug delivery device 501 described and illustrated in reference to FIGS. 8A-8C, the drug delivery device 401 described and illustrated in reference to FIG. 7, the drug delivery device 301 described and illustrated in reference to FIGS. 6A-6C, the drug delivery device 201 described and illustrated in reference to FIG. 5, the drug delivery device 101 described and illustrated in reference to FIG. 4, the drug delivery device 31 described and illustrated in reference to FIG. 3, and the drug delivery device 1 described and illustrated in reference to FIG. 1A-2M may all be interchangeable.

Each example of an adhesive platform/adhesive platform components as described herein may be interchangeable with adhesive platforms/adhesive platform components described in other examples or embodiments herein. For example, the adhesive platform 1705 described and illustrated in reference to FIG. 17, the adhesive platform 1605 described and illustrated in reference to FIG. 16, the adhesive platform 1505 described and illustrated in reference to FIGS. 15A-15F, the adhesive platform components 1405a and 1405b described and illustrated in reference to FIGS. 15A and 14B, the adhesive platform 1305a and 1305b described and illustrated in reference to FIG. 13, adhesive platform 1205 described and illustrated in reference to FIG. 12, the adhesive platform 1105 described and illustrated in reference to FIG. 11, the adhesive platform 1005 described and illustrated to FIGS. 10A-10C, the adhesive platform 905 described and illustrated in reference to FIGS. 9A and 9B, the adhesive platform 505 described and illustrated in reference to FIGS. 8A-8C, the adhesive platform 405 described and illustrated in reference to FIG. 7, the adhesive platform 305 described and illustrated in reference to FIGS. 6A-6C, the adhesive platform 205 described and illustrated in reference to FIG. 5, the adhesive platform 105 described and illustrated in reference to FIG. 4, the adhesive platform 45 described and illustrated in reference to FIG. 3, and the adhesive platform 5 described and illustrated in reference to FIG. 1A-2M may all be interchangeable.

Each example of a temporary backing/temporary backing components as described herein may be interchangeable with temporary backings/temporary backing components described in other examples or embodiments herein. For example, the temporary backing 1706 described and illustrated in reference to FIG. 17, the temporary backing 1606 described and illustrated in reference to FIG. 16, the temporary backing 1506 described and illustrated in reference to FIGS. 15A-15F, the temporary backing components 1406a and 1406b described and illustrated in reference to FIGS. 15A and 14B, the temporary backing 1306a and 1306b described and illustrated in reference to FIG. 13, temporary backing 1206 described and illustrated in reference to FIG. 12, the temporary backing 1106 described and illustrated in reference to FIG. 11, the temporary backing 1006 described and illustrated to FIGS. 10A-10C, the temporary backing 906 described and illustrated in reference to FIGS. 9A and 9B, the temporary backing 506 described and illustrated in reference to FIGS. 8A-8C, the temporary backing 406 described and illustrated in reference to FIG. 7, the temporary backing 306 described and illustrated in reference to FIGS. 6A-6C, the temporary backing 206 described and illustrated in reference to FIG. 5, the temporary backing 106 described and illustrated in reference to FIG. 4, and the temporary backing 6 described and illustrated in reference to FIGS. 1A-2M may all be interchangeable.

Each example of temporary a backing tab/temporary backing tab first and second components as described herein may be interchangeable with temporary backing tab/temporary backing tab first and second components described in other examples or embodiments herein. For example, the temporary backing tab first and second components 1706a and 1706b described and illustrated in reference to FIG. 17, the temporary backing tab first and second components 1604a and 1604b described and illustrated in reference to FIG. 16, the temporary backing tab first and second components 1504a and 1504b described and illustrated in reference to FIGS. 15A-15F, the temporary backing tab first and second components 1304a and 1304b described and illustrated in reference to FIG. 13, temporary backing tab first and second components 1204a and 1204b described and illustrated in reference to FIG. 12, the temporary backing tab first and second components 1104a and 1104b described and illustrated in reference to FIG. 11, the temporary backing tab first and second components 1004a and 1004b described and illustrated to FIGS. 10A-10C, the temporary backing tab first and second components 904a and 904b described and illustrated in reference to FIGS. 9A-9B, the temporary backing tab first and second components 504a and 504b described and illustrated in reference to FIGS. 8A-8C, the temporary backing tab first and second components 404a and 404b described and illustrated in reference to FIG. 7, the temporary backing tab first and second components 304a and 304b described and illustrated in reference to FIGS. 6A-6C, the temporary backing tab 34 described and illustrated in reference to FIG. 3, and the temporary backing tab 4 described and illustrated in reference to FIGS. 1A-2M may all be interchangeable.

Each example of first and second temporary wings as described herein may be interchangeable first and second temporary wings described in other examples or embodiments herein. For example, the temporary wings 508 and 509 described and illustrated in reference to FIGS. 8A-8C, the temporary wings 508 and 509 described and illustrated in reference to FIG. 7, the temporary wings 308 and 309 described and illustrated in reference to FIGS. 6A-6C, the temporary wings 38 and 39 described and illustrated in reference to FIG. 4, and the temporary wings 8 and 9 described and illustrated in reference to FIGS. 1A-2M may all be interchangeable.

Each example of a movable layer as described herein may be interchangeable with movable layers/movable layer components described in other examples or embodiments herein. For example, the movable layer 1760 described and illustrated in reference to FIG. 17, the movable layer 1660 described and illustrated in reference to FIG. 16, the movable layer 1560 described and illustrated in reference to FIGS. 15A-15F, the movable layer components 1460a and 1460b described and illustrated in reference to FIGS. 14A and 14B, the movable layer components 1360a and 1360b described and illustrated in reference to FIG. 13, the movable layer components 1260a and 1260b described and illustrated in reference to FIG. 12, the movable layer components 1160a and 1160b described and illustrated in reference to FIG. 11, and the movable layer 1060 described and illustrated to FIGS. 10A-10C may all be interchangeable.

Each example of a first temporary handle and second temporary handle as described herein may be temporary handles described in other examples or embodiments herein. For example, the first temporary handle 1720 and second temporary handle 1721 described and illustrated in reference to FIG. 17, the first temporary handle 1620 and second temporary handle 1621 described and illustrated in reference to FIG. 16, the first temporary handle 1520 and second temporary handle 1521 described and illustrated in reference to FIGS. 15A-15F, the first temporary handle 1420 and second temporary handle 1421 described and illustrated in reference to FIGS. 14A and 14B, the first temporary handle 1320 and second temporary handle 1321 described and illustrated in reference to FIG. 13, the first temporary handle 1220 and second temporary handle 1221 described and illustrated in reference to FIG. 12, the first temporary handle 1120 and second temporary handle 1121 described and illustrated in reference to FIG. 11, and the first temporary handle 1020 and second temporary handle 1021 described and illustrated to FIGS. 10A-10C may all be interchangeable.

What is claimed is:

1. A medical device application system for positioning a medical device on a user, the medical delivery application system comprising:
an adhesive platform operable to be coupled to the medical device, the adhesive platform comprising a first platform surface and a second platform surface;
a temporary backing detachably connected to the first platform surface of the adhesive platform, wherein the second platform surface of the adhesive platform is configured to interface with the wearable drug delivery device; and
a first temporary backing wing extending from a first backing side of the temporary backing and a second temporary backing wing extending from a second side of the temporary backing, wherein the second backing side extends from the opposite side as the first backing side.

2. The medical device application system of claim 1, further comprising a temporary backing tab extending from the temporary backing.

3. The medical device application system of claim 1, wherein the adhesive platform further comprises:
a first platform bridge extending from a first platform side of the temporary backing, and a second platform bridge extending from a second platform side of the temporary backing, wherein the second side is opposite the first side, and
the adhesive platform bridges are paired with the temporary backing bridges.

4. The medical device application system of claim 1, wherein the medical device is a drug delivery device.

5. The medical device application system of claim 1, wherein the medical device is an analyte sensor.

6. The medical device application system of claim 1, wherein the adhesive platform is configured to adhere to skin of the user.

7. The medical device application system of claim 1, further comprising a temporary loop detachably connected to an external facing surface of the medical device.

8. The medical device application system of claim 7, wherein:
the temporary loop has a first end and a second end that are detachably adhered to the external facing surface of the medical device.

9. The medical device application system of claim 8, further comprising a temporary loop tab extending from the first end of the temporary handle.

10. The medical device application system of claim 8, wherein the temporary loop is configured to permit one or more human fingers to pass between an opening in the temporary handle and the external facing surface of the medical device.

11. The medical device application system of claim 1, further comprising a first sticker having a first sticker adhesive side and a first sticker gripping side; and
a second sticker having a second sticker adhesive side and a second sticker gripping side, wherein:
at least a first portion of the first sticker adhesive side and at least a first portion of the second sticker adhesive side are detachably connected to the medical device external facing surface, and
a second portion of the first sticker adhesive side is detachably connected to a second portion of the second sticker adhesive side.

12. The medical device application system of claim 11, wherein the detachably connected portions of the first sticker and the second sticker are configured to form a temporary handle.

13. A method of assembling a medical device application system for positioning a medical device on a user, comprising:
covering, at least partially, an internal facing side of an adhesive platform with an adhesive;
placing a temporary backing on top of the internal facing side of the adhesive platform;
cutting the temporary backing attached to the adhesive platform into a shape;
disposing the adhesive on an internal facing surface of a medical device or the adhesive platform; and
attaching the drug delivery device to the external facing surface of the adhesive platform.

14. The method of claim 13, wherein the shape is a shape of the internal facing surface of the medical device.

15. The method of claim 13, further comprising:
covering at least a portion of the external facing side of the adhesive platform with adhesive; and
attaching a temporary backing first wing and a temporary backing second wing to the adhesive platform.

16. The method of claim 15, further comprising moving an internal facing surface of the temporary backing first wing and an internal facing surface of the temporary backing second wing towards each other and away from the internal facing surface of the medical device.

17. The method of claim 13, further comprising holding the drug delivery device with a temporary handle so that a temporary backing and a temporary backing tab are exposed; and
pulling the temporary backing by the temporary backing tab in a direction perpendicular to a patient facing surface of the medical device.

18. The method of claim 13, further comprising applying the medical device to an abdomen, thigh, or arm of a user.

19. The method of claim 13, wherein the medical device is a drug delivery device.

20. The method of claim 13, wherein the medical device is an analyte sensor.
